(19) United States
(12) Patent Application Publication
(10) Pub. No.: US 2026/0056050 A1
(43) Pub. Date: Feb. 26, 2026

(54) MONITORING THE BRIGHTNESS OF A LIGHT SOURCE OF A LIGHT SYSTEM

(71) Applicant: Alcon Inc., Fribourg (CH)

(72) Inventors: Annelene Konz, Hollfeld (DE); Joerg Michael Hurich, Lauf an der Pegnitz (DE)

(21) Appl. No.: 19/295,813

(22) Filed: Aug. 11, 2025

Related U.S. Application Data
(60) Provisional application No. 63/684,909, filed on Aug. 20, 2024.

(51) Int. Cl. G01J 1/42; A61B 3/00; A61B 3/14; G06T 7/00; G06T 7/90

(52) U.S. Cl. CPC G01J 1/42; A61B 3/0041; A61B 3/14; G06T 7/0012; G06T 7/90; G01J 2001/4247; G06T 2207/10024; G06T 2207/30041

(57) ABSTRACT

In certain embodiments, an ophthalmic system includes a light system, an imaging system, and a computer. The light system directs light towards a test target according to an expected parameter set to yield an actual spot on the test target. Each expected brightness parameter of the set describes an expected brightness of the actual spot. The imaging system generates a digital image of the actual spot. The computer determines pixel values of the pixels of the digital image and determines an actual brightness parameter set according to the pixel values. Each actual brightness parameter of the set describes an actual brightness of the actual spot and corresponds to an expected brightness parameter. The computer compares the actual brightness parameters to the corresponding expected brightness parameters and detects a deviation. The computer identifies an issue of the light system indicated by the deviation and provides an output associated with the issue.

### FIELD

Embodiments of the present disclosure relate to monitoring the brightness of a light source of a light system.

### BACKGROUND

Light sources play an important role in, e.g., ophthalmological laser systems. They can be used to illuminate the target as well as provide auxiliary light for other purposes. For example, an auxiliary light may be used to visualize the path of a laser beam or to guide a patient's gaze for surgery.

### SUMMARY

In certain embodiments, an ophthalmic system includes a light system, an imaging system, and a computer. The light system directs light towards a test target located at a target plane according to an expected parameter set to yield an actual spot on the test target. The expected parameter set includes one or more expected brightness parameter(s), where each expected brightness parameter describes an expected brightness of the actual spot. The imaging system includes one or more digital camera(s) that can generate a digital image of the actual spot. The computer determines pixel values of the pixels of the digital image and determines an actual brightness parameter set according to the pixel values. The actual brightness parameter set includes one or more actual brightness parameter(s). Each actual brightness parameter describes an actual brightness of the actual spot and corresponds to an expected brightness parameter. The computer compares each actual brightness parameter to the corresponding expected brightness parameter and detects one or more deviation(s) of the actual brightness parameter(s) from the expected brightness parameter(s) in response to the comparison. The computer identifies one or more issue(s) of the light system indicated by the deviation(s) and provides an output associated with the issue(s) of the light system.

Embodiments may include one, two, more, all, or any combination of the following.
The computer determines the actual brightness parameter set according to the pixel values by: selecting actual spot pixel values from the pixel values, where the actual spot pixel values represent the actual spot, and determining the actual brightness parameter set using the actual spot pixel values. The computer may select the actual spot pixel values from the pixel values by identifying the actual spot pixel values according to an actual spot range of pixel values. The computer may select the actual spot pixel values from the pixel values by identifying the actual spot pixel values according to a user selection.
The actual brightness parameter set includes one or more elements selected from a group comprising: a maximum grayscale value, an actual maximum brightness value, an average grayscale value, an actual average brightness value, a grayscale statistical distribution value, an actual brightness statistical distribution value, a grayscale distribution evenness value, and an actual brightness distribution evenness value.
The pixel values comprise grayscale values. The computer may determine the actual brightness parameter set according to the pixel values by identifying a maximum grayscale value of the grayscale values and determining an actual maximum brightness value according to the maximum grayscale value. The computer may determine the actual brightness parameter set according to the pixel values by calculating an average grayscale value of the grayscale values and determining an actual average brightness value according to the average grayscale value. The computer may determine the actual brightness parameter set according to the pixel values by calculating a grayscale statistical distribution value of the grayscale values and determining an actual brightness statistical distribution value according to the grayscale statistical distribution value. The computer may determine the actual brightness parameter set according to the pixel values by calculating a grayscale distribution evenness value of the grayscale values and determining an actual brightness distribution evenness value according to the grayscale distribution evenness value.
The pixel values include color scale values, and the computer determines an actual color value according to at least one color scale value. The computer may determine the actual color value by calculating an average color scale value of at least two color scale values. The computer may determine the actual color value by calculating a color scale distribution evenness value of at least two color scale values.
The computer provides the output associated with the issue(s) of the light system by calculating a correction to compensate for a deviation of the deviation(s) and instructing the light system to implement the correction to compensate for the deviation of the deviation(s).
The computer provides the output associated with the issue(s) of the light system by providing a notification of the difference in brightness indicated by the issue(s).
The computer provides the output associated with the issue(s) of the light system by providing a notification of a difference in color indicated by the issue(s).
The computer provides the output associated with the issue(s) of the light system by providing a warning notification if an actual brightness parameter of the actual brightness parameter(s) satisfies a warning range.
The computer provides the output associated with the issue(s) of the light system by providing a notification to a user via an interface.
The computer provides the output associated with the issue(s) of the light system by sending a notification to a service technician.
The computer records an actual brightness parameter in a historical data set comprising historical actual brightness parameters. The computer may analyze the historical data set to determine a warning range indicating a range of actual brightness values that initiate a warning associated with the light system. The computer may analyze the historical data set to determine an average lifespan of the light system and provide a reminder to check the light system prior to the expiration of the average lifespan of the light system. The computer may analyze the historical data set to detect a trend of an issue of the light system.
The computer performs the following each known brightness value of a set of known brightness values to yield a set of calibration pixel values, where each calibration pixel value corresponds to a known brightness value: instruct the light system to direct light towards a calibration target located at the target plane at each known brightness value to yield a calibration spot on the calibration target; instruct the imaging system to generate a digital calibration image of the calibration spot at each known brightness value, the digital calibration image comprising calibration pixels; and analyze the calibration pixels to determine the set of calibration pixel values. The computer may determine a mathematical relationship between the calibration pixel values and the known brightness values.
The light system includes at least one system selected from a group comprising: a laser system that can direct a laser beam towards the target plane; an illumination light system that can direct an illumination beam towards the target plane to illuminate the target plane; an aiming light system that can direct an aiming beam towards the target plane to align a laser beam and the target plane; and a fixation light system that can direct a fixation beam towards the target plane to align an eye and a laser system.

In certain embodiments, an ophthalmic system includes an imaging system and a computer. The imaging system includes one or more digital cameras that can generate a digital image of an actual eye. The computer determines pixel values of the pixels of the digital image and determines an actual color parameter set according to the pixel values. The actual color parameter set includes one or more actual color parameter(s), where each actual color parameter describes an actual color of the actual eye. The computer compares each actual color parameter to a corresponding expected color parameter of one or more expected color parameter(s), where each expected color parameter describes an expected color of an expected eye, and detects one or more deviation(s) of the actual color parameter(s) from the expected color parameter(s) in response to the comparison. The computer identifies one or more issue(s) indicated by the deviation(s) and provides an output associated with the issue(s).

Embodiments may include one, two, more, all, or any combination of the following.
The computer identifies the issue(s) by determining that the actual eye is not the expected eye. The computer may provide the output by providing a notification that the actual eye is not the expected eye.
The computer identifies the issue(s) by determining that the actual eye has a medical condition. The computer may provide the output by providing a notification that the actual eye has the medical condition.

### BRIEF DESCRIPTION OF THE DRAWINGS

FIG. 1 illustrates an example of a system configured to detect an issue with a light system, according to at least one embodiment described in the present disclosure;

FIG. 2 illustrates an example of a computer that may be used with a system configured to detect an issue with a light system, according to at least one embodiment described in the present disclosure;

FIG. 3 illustrates an example computer system, according to at least one embodiment described in the present disclosure;

FIGS. 4A and 4B illustrate an example output that describes an actual spot in a digital image, according to at least one embodiment described in the present disclosure;

FIGS. 5A and 5B illustrate another example output that describes an actual spot in a digital image, according to at least one embodiment described in the present disclosure;

FIG. 6 illustrates an example of a method of detecting an issue with a light source, according to at least one embodiment described in the present disclosure;

FIG. 7 illustrates an example of a calibration method, according to at least one embodiment described in the present disclosure; and

FIG. 8 illustrates an example of detecting an issue with an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Certain known ophthalmic laser systems do not monitor the brightness of the light sources of their light systems. Typically, the brightness of a light source is adjusted during manufacturing using an external measurement device. The brightness may be rechecked during servicing by a service technician with an external measurement device. However, there typically is not regular, periodic, or continual monitoring of the brightness of the light source.

The present disclosure relates to monitoring the brightness of a light source of a light system. According to certain embodiments, the light source directs light towards a test target according to expected parameters to yield an actual spot on the test target. A digital image of the actual spot is analyzed to detect differences between the actual spot and the expected parameters. For example, the analysis may detect a change in brightness, which may indicate degradation of the light source.

Certain embodiments of the present disclosure may provide improvements over known iterations of monitoring the brightness of a light source of a light system. For example, a system may automatically check the brightness of a light source and may adjust the brightness and/or may provide notification of an issue with the light source. As another example, a system may check the light source without requiring a service technician or an external measuring device. As yet another example, a system may check the light source at any suitable time, e.g., right after manufacturing the device, while servicing the device, and/or before performing a procedure. As yet another example, a system may detect degradation of the light source and provide a warning. As yet another example, a system may analyze historical data to determine a trend in an issue with the light source. As yet another example, a system may check the color of a patient's eye to detect a medical condition.

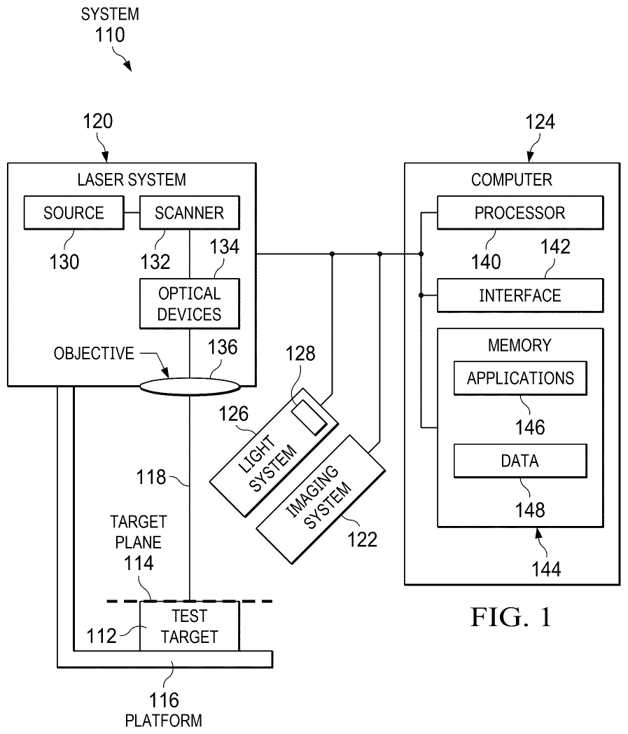

FIG. 1 illustrates an example of a system 110 configured to detect an issue with a light system 126, according to at least one embodiment described in the present disclosure. In the example, the system 110 uses a test target 112 located at a target plane 114 to detect issues with the light system 126. The system 110 includes a platform 116, a laser system 120, an imaging system 122, a computer 124, and the light system 126 (with a light source 128), which may be coupled as shown. The laser system 120 includes a laser source 130, a scanner 132, optical devices 134, and an objective 136, which may be coupled as shown. The computer 124 includes a processor 140, an interface 142, and a memory 144, which stores applications 146 and data 148, which may be coupled as shown.

According to an example of operation, the light system 126 directs light towards the test target 112 located at the target plane 114 according to an expected parameter set to yield an actual spot on the test target 112. The expected parameter set includes one or more expected brightness parameters, where each expected brightness parameter describes an expected brightness of the actual spot. The imaging system 122 includes one or more digital cameras that can generate a digital image of the actual spot.

In the example, the computer 124 analyzes the digital image to determine pixel values of the pixels of the digital image and selects a set of the pixel values that includes pixel values that represent the actual spot. The computer 124 determines an actual brightness parameter set using the set of the pixel values. The actual brightness parameter set includes actual brightness parameters that each describe an actual brightness of the actual spot. Each actual brightness parameter corresponds to an expected brightness parameter. The computer 124 compares each actual brightness parameter to the corresponding expected brightness parameter and detects a deviation of the actual brightness parameters from the expected brightness parameters. The computer 124 identifies an issue of the light system indicated by the deviation and provides output in response to the issue of the light system.

According to another example of operation, the imaging system 122 generates a digital image of the eye. The computer 124 determines the pixel values of the pixels of the digital image and then determines an actual color parameter set according to the pixel values. The actual color parameter set includes actual color parameters describing the actual color of the actual eye. The computer 124 compares each actual color parameter to a corresponding expected color parameter that describes an expected color of an expected eye and detects a deviation of the actual color parameters from the expected color parameters. The computer 124 identifies an issue indicated by the deviation and provides an output corresponding to the issue.

For ease of explanation, the embodiments are described using the following example xyz-coordinate system, which may be regarded as the coordinate system of the system 110, although any suitable coordinate system may be used. In the example, the z-axis is aligned with the optical axis of the laser system 120, and the xy-plane is orthogonal to the z-axis and may be located at, e.g., the target plane 114.

The System. The system 110 may be any suitable medical device that performs a medical procedure, such as a surgical and/or diagnostic procedure. In certain embodiments, the system 110 may be a surgical system that performs surgical procedures on humans, such as an ophthalmic surgical system that performs surgical procedures on human eyes. Examples of laser surgical systems include cataract, refractive, vitreoretinal, or other ophthalmic surgical system.

Test Target. Turning to the components of FIG. 1, the test target 112 is located at the target plane 114 and may be supported by the platform 116. In certain embodiments, the target plane 114 may represent the treatment plane and may designate the z=0 plane. For example, the treatment plane indicates where the laser spots of laser system 20 will be directed, e.g., a surgical site of human tissue such as the eye. Or stated another way, the distance from the laser system 120 to the target plane 114 may correspond to an expected or average distance from the laser system 120 to the surgical site when performing the surgery on human tissue (e.g., an eye).

In certain embodiments, the test target 112 comprises a photosensitive material or sensor that undergoes a visible change where a light beam interacts with the test target 112 to indicate where the light beam interacted with the test target 112. The visible change may be a change in color, where “color” includes chromatic and achromatic (e.g., grayscale) colors. For example, the material may be one color (a “non-radiated color”) if there is no interaction, but may change to another color (an “irradiated color”) if light interacts with it. Examples of the test target 12 include photographic paper, metal foil, conversion screens, and polymethyl methacrylate (PMMA) material. In certain embodiments, the test target 112 may include a thin paper or material that may form a hole or void in the material when contacted by a light beam, and the hole may be representative of a change in color. In certain embodiments, the test target 112 may comprise material that reflects a light beam to yield reflection indicating where the light beam was reflected. In certain embodiments, the test target 112 may comprise a material (e.g., human tissue such an eye) that reflects light such that the imaging system 122 can generate an image of the test target 112.

Laser System. In the example, the laser system 120 directs a laser beam 118 towards the target plane 114 to, e.g., perform a surgical procedure. More specifically, the laser source 130 generates the laser beam 118, and the scanner 132 guides the beam 118. One or more optical devices 134 direct the laser beam 118 towards the focusing objective 136, which directs the laser beam 118 towards the test target 112. Examples of the laser source 130 include excimer and femtosecond lasers. The laser beam 118 may have any suitable pulse duration, such as in the order of nanoseconds, picoseconds, femtoseconds, or attoseconds. The laser beam 118 may have any suitable wavelength, such as in the range of 150 nanometers (nm) to 20 micrometers (μm). Examples of ranges include the ultraviolet (e.g., in the range of 180 to 400 nm, such as 190 to 195 nm or 345 to 355 nm), visible, or infrared wavelength (e.g., in the range of 1050 to 1250 or 1250 to 1500 nm). The laser beam 118 may process material in any suitable manner, e.g., ablate, incise, or photo-disrupt the material.

Imaging System. The imaging system 122 includes one or more digital cameras or other optical sensors that can generate a digital image of an actual spot on the test target 112. In general, a digital camera or other optical sensor detects light from an object and generates a signal in response to the light. The signal carries digital image data that can be used to generate the digital image of the object. Examples of digital cameras include a charged-coupled device (CCD) camera, a video camera, a complementary metal-oxide semiconductor (CMOS) sensor (e.g., active-pixel sensor (APS)), a line sensor, and an optical coherence tomography (OCT) camera. In certain embodiments, the imaging system 122 includes a digital microscope with one, two, or more cameras that can provide a magnified image of the test target 112. In certain embodiments, the imaging system 122 may have one or more filters, e.g., a color filter and/or an edge filter. A filter may filter the light that enters the imaging system 122.

Light System. The light system 126 directs light (e.g., a laser beam, illumination, or other light) towards the test target 112 according to an expected parameter set to yield an actual spot on the test target 112 that may be used to detect issues with the light system 126. The spot may be any suitable size or shape, i.e., the spot is not necessarily circular. The expected parameter set includes one or more expected brightness parameters, and each expected brightness parameter describes an expected brightness of the actual spot. Examples of brightness parameters are described herein. The light system 126 may have any suitable light source 128, such as a light source that produces light via incandescence (e.g., a bulb or lamp) and/or electroluminescence (e.g., a light-emitting diode).

In certain embodiments, the light system 126 may be the laser system 120, and the brightness of the laser beam emitted by the laser system 120 may be monitored. In certain embodiments, the light system 126 may be an auxiliary light system that emits an auxiliary light (e.g., an aiming beam, a fixation light, an illumination light, and/or one or more distance beams), and the brightness of the auxiliary light may be monitored. For example, the light system 126 may be an illumination light system that directs an illumination beam towards the target plane 114 to illuminate a target. As another example, the light system 126 may be an aiming light system that directs an aiming beam towards the target plane 114 to indicate the location of a laser spot to align the laser system 120 with a target. As another example, the light system 126 may be a distance beam system that creates distance beams that indicate distance in the z-direction, e.g., the spots formed by the beams overlap when the target plane 114 is at the correct z-position. As another example, the light system 126 may be a fixation light system that directs a fixation beam towards the target plane to align an eye with a laser system.

Computer. The computer 124 performs operations to monitor the brightness of a light source 128, including sending instructions to other components of the system 110 (e.g., the laser system 120, the imaging system 22, and/or the light system 126) to perform operations. In the example, the computer 124 analyzes a digital image of an actual spot created by the light source 128 to determine the actual brightness of the actual spot. The computer 124 compares the actual brightness to the expected brightness to detect a deviation of the actual brightness from the expected brightness. The computer 124 identifies an issue of the light system indicated by the deviation and provides an output describing the issue.

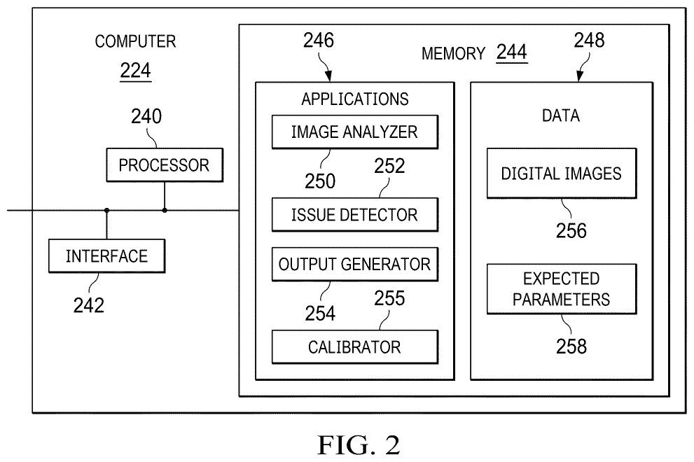

FIG. 2 illustrates an example of a computer 224 that may be used with a system configured to detect an issue with a light system, according to at least one embodiment described in the present disclosure. In the example, the computer 224 includes a processor 240, an interface 242, and a memory 244, which stores applications 246 and data 248. Applications 246 include an image analyzer 250, an issue detector 252, an output generator 254, and/or a calibrator 255. Data 248 include digital images 256 and/or expected parameters 258.

Data. The digital images 256 include images of actual spots on a test target. The digital images 256 may also include calibration images used to calibrate brightness levels. In certain cases, a digital image may be a composite of multiple images taken from different cameras and/or taken at different times. An expected parameter 258 instructs a light system to direct light towards a test target using a specific setting, e.g., a specific brightness setting.

Image Analysis. In certain embodiments, the image analyzer 250 uses image processing to analyze pixel values (e.g., grayscale and/or color scale values) of digital images. The grayscale value of a pixel represents the brightness or intensity value of the pixel and may be expressed in any suitable manner. For example, a 0% to 100% scale may be used, where 0% represents total absence, black, and 100% represents total presence, white. As another example, a 0 to 255 scale may be used, where 0 represents total absence and 255 represents total presence. The color scale value of a pixel represents the color of the pixel and may be expressed in any suitable manner, such as a transformation of three colors such as red, green, and blue.

In certain embodiments, the image analyzer 250 selects actual spot pixel values representing the actual spot from the pixel values of the digital image and determines an actual brightness parameter set from actual spot pixel values. The image analyzer 250 may select the actual spot pixel values in any suitable manner. In certain embodiments, the image analyzer 250 may identify which pixel values are actual spot pixel values according to a range of pixel values expected of the actual spot. For example, pixel values of an irradiated color (e.g., a lighter color or a white color) may represent the actual spot. In some cases, pixel values can indicate the degree of irradiation, such that some pixel values indicate more irradiation and other pixel values indicate less irradiation. As another example, the actual spot may be expected to have a brightness in a range of, e.g., greater than 70% brightness, and/or the actual spot may be expected to have a color in a yellow and/or white range. In certain embodiments, the image analyzer 250 may identify the actual spot pixel values according to a user selection. For example, the user may manipulate a graphical element to select pixels representing the actual spot, as described herein.

In certain embodiments, the image analyzer 250 determines a set of actual brightness parameters from the actual spot pixel values in any suitable manner. In certain embodiments, the image analyzer 250 calculates the actual brightness of at least a portion of an actual spot using a mathematical relationship between the brightness values and pixel values of the digital image. For example, the mathematical relationship may be a mathematical function f(P)=B, where P represents a pixel value and B represents a brightness value. The mathematical relationship may have any suitable form. For example, the relationship may translate a pixel value to a brightness value expressed in a unit of brightness (e.g., lumens). As another example, the relationship may yield a brightness value that is essentially the same as the given pixel value, e.g., the pixel value may be used to describe brightness. A calibration process to determine a mathematical function is described herein.

In certain embodiments, the image analyzer 250 may determine an actual brightness parameter according to grayscale values that represent at least a portion of the actual spot. For example, the image analyzer 250 may identify a maximum grayscale value of the grayscale values and determine an actual maximum brightness value according to the maximum grayscale value. As another example, the image analyzer 250 may calculate the average grayscale value of the grayscale values of the area of at least a portion of the actual spot and determine the actual average brightness value according to the average grayscale value.

As another example, the image analyzer 250 may calculate the statistical distribution of the grayscale values and determine the actual statistical distribution of brightness values according to the statistical distribution of grayscale values. The statistical distribution of the grayscale values and/or the brightness values may be, e.g., a frequency distribution of the grayscale values and/or the brightness values. In yet another example case, the image analyzer 250 may calculate a grayscale distribution evenness value of the grayscale values and determine an actual brightness distribution evenness value according to the grayscale distribution evenness value. The grayscale distribution evenness value may be a color distribution evenness (CDE) metric that measures the constancy of color or grayscale values. The brightness distribution evenness values may be proportional to the grayscale distribution evenness values, such that a greater grayscale distribution evenness value yields a greater brightness distribution evenness value.

In certain embodiments, the image analyzer 250 may determine an actual brightness parameter according to color scale values of pixels that represent at least a portion of the actual spot. For example, the image analyzer 250 may determine an actual color value according to at least one color scale value. An actual color value may be an average color scale value or a color scale distribution evenness value calculated from color scale values.

Issue Detection and Output Generation. In certain embodiments, the issue detector 252 compares the actual parameters to the expected parameters, detects deviations of the actual parameters from the expected parameters, and identifies issues indicated by the deviations. A deviation may be a difference greater than a predetermined margin of error, which may be a value within a range of 1 to 10 percent, e.g., 2 to 5, 5 to 10, and/or 10 to 20 percent. In certain embodiments, the expected and/or actual parameters may be converted to the same or similar formats so the issue detector 252 can compare the parameters. The deviations may indicate certain issues with a light source or a patient eye, as described herein.

The output generator 254 generates and provides output in response to an identified issue. The output may be a notification provided any suitable sensory format, e.g., visual, auditory, and/or tactile. The output may be provided in any suitable manner, e.g., the output may be provided via a user interface to the user, via a computer instruction to a component of a system, and/or via a communication network to a service technician. The output may include information described in any suitable manner, e.g., alphanumeric characters (such as text), images (such as photographs or videos), audio, and/or graphs.

The output generator 254 may provide any suitable output in response to an identified issue. For example, the issue detector 252 may detect a difference in brightness and/or color, and the output generator 254 may output a notification describing the difference. As another example, the issue may be that an actual brightness value satisfies a warning range, and the output may be a warning notification. As another example, the issue detector 252 detects a deviation indicating that the actual eye is not the expected eye, and the output generator 254 provides a notification that the actual eye is not the expected eye. As another example, the issue detector 252 detects a deviation indicating that the actual eye has a medical condition, and the output generator 254 provides a notification that the actual eye has a medical condition.

In certain embodiments, the output may be a command sent to a light system in response to detecting a problem. For example, the command may include instructions to the light system to correct a detected problem, prevent a laser system from generating a laser beam, and/or shut off the laser system. In an example case, the output generator 254 can calculate a correction to compensate for a deviation and generate a command to instruct the light system to implement the correction. For example, the output generator 254 may determine that the light system is generating a light that is X% too dim, so the correction may be to generate a light that is X% brighter in order to regulate the lighting.

In certain embodiments, the issue detector 252 can detect trends in issues over time. In the embodiments, the issue detector 252 accesses and analyzes images of actual spots taken over, e.g., a number of weeks, months, or years, to detect a trend of an issue with the laser system. For example, the issue detector 252 may detect that a light system tends to become dimmer after a certain amount of time, e.g., X number of months. The output generator 254 may provide an output that notifies a user of potential decreased illumination during a warning period prior to the certain number of months. As another example, the issue detector 252 may analyze the historical data set to determine an average lifespan of the light source, and output generator 254 may provide a reminder to check the light system prior to an expiration of the average lifespan. As another example, the issue detector 252 analyzes a historical data set that records actual brightness parameters gathered during previous iterations. From the analysis, the issue detector 252 may determine a warning range of actual brightness values that precede an issue with the light system. The output generator 254 may provide a warning if an actual brightness value is in the warning range. As another example, the issue detector 252 analyzes illumination during initialization of the system 110 to determine if the illumination satisfies initialization requirements, e.g., the illumination should reach a particular value during initialization. If the illumination satisfies the requirements, the output generator 254 may provide notification that the system 110 has been initialized.

Calibration. The calibrator 255 performs operations to calibrate the brightness detection. In certain embodiments, the calibrator 255 accesses a set of known brightness values and instructs the light system to direct light towards a calibration target located at the target plane at a known brightness value to yield a calibration spot. The calibrator 255 instructs an imaging system to generate a digital calibration image of the calibration spot and analyzes the calibration pixels to determine a calibration pixel value that corresponds to the known brightness value.

In certain embodiments, the calibrator 255 may determine a mathematical relationship between the calibration pixel values and the known brightness values. For example, the computer may perform a least-squares fit of the values to yield a mathematical function f(P)=B that yields a brightness value B for a given pixel value P. In certain embodiments, the calibrator 255 may determine a range of calibration pixel values that correspond to a range of known brightness values. For example, the computer may determine a range of calibration pixel values that correspond to an expected range of brightness values. As another example, the computer may determine a range of calibration pixel values that correspond to a warning range of brightness values that may initiate a warning by the system.

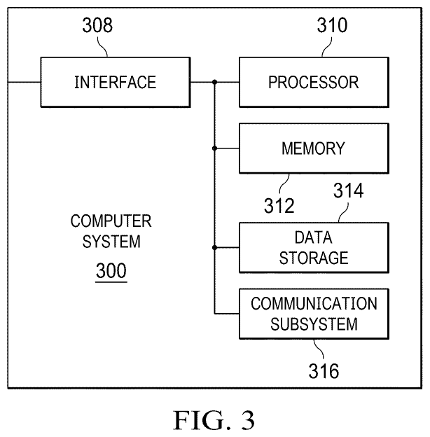

FIG. 3 illustrates an example computer system 300, according to at least one embodiment described in the present disclosure. The computer system 300 may include an interface 308, a processor 310, a memory 312, a data storage 314, and/or a communication subsystem 316, any or all of which may be communicatively coupled. Any or all of the computer system 300 may be implemented as computer hardware and/or software. Any or all of the systems described herein may be implemented as a computer system consistent with the computer system 300.

In the example, the interface 308 may receive input to the computer system 300 and/or send output from the computer system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computer system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computing entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

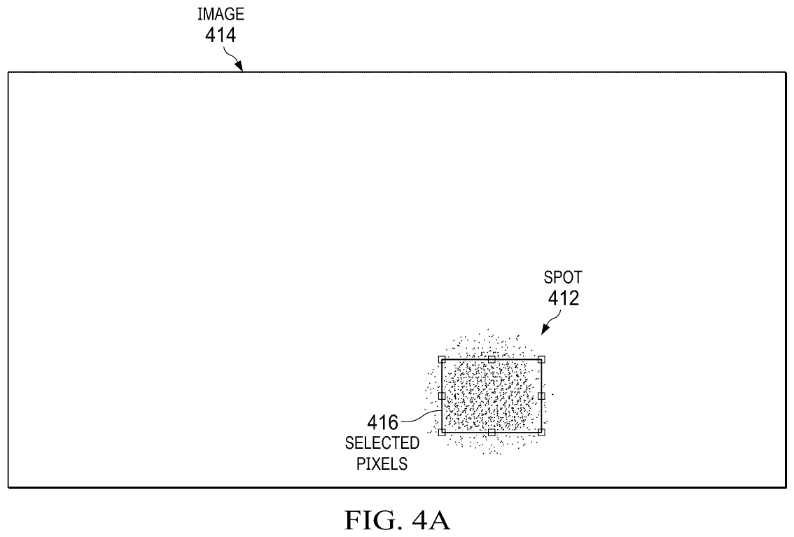

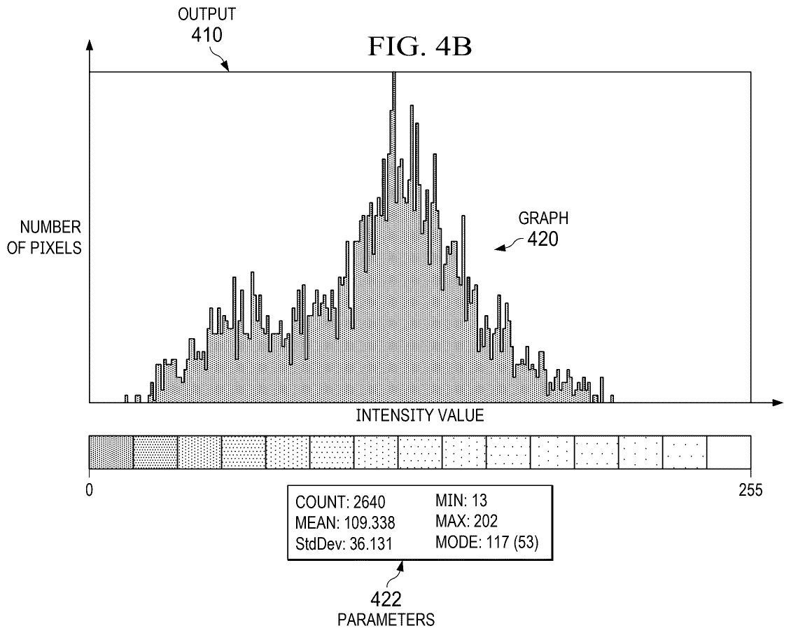

FIGS. 4A and 4B illustrate an example output 410 that describes an actual spot 412 in a digital image 414, according to at least one embodiment described in the present disclosure. FIG. 4A shows the digital image 414 with the spot 412 formed where light from a light system interacted with a target. The digital image 414 shows selected pixels 416 that were selected to be analyzed. The selected pixels may be automatically selected by a computer or may be selected by the user. For example, the computer may automatically select the pixels based on the expected size of the spot 412. As another example, the user may manipulate the graphical element representing the bounding box of the selected pixels 416 in order to select the pixels.

FIG. 4B shows the output 410 of an analysis of the brightness of a light source of the light system. The output 410 includes a graph 420 and actual parameters 422. The graph 420 shows a frequency distribution of the brightness values of the selected pixels 416. The graph 420 has an x-axis that represents pixel intensity values (e.g., a range from 0 to 255) and a y-axis that represents the frequency count of a number of pixels. The actual parameters 422 may include the total number of pixels (COUNT), the minimum pixel value (MIN), the maximum pixel value (MAX), and mean pixel value (MEAN), the standard deviation of the pixel values (StdDev), and the mode of the pixel values (MODE).

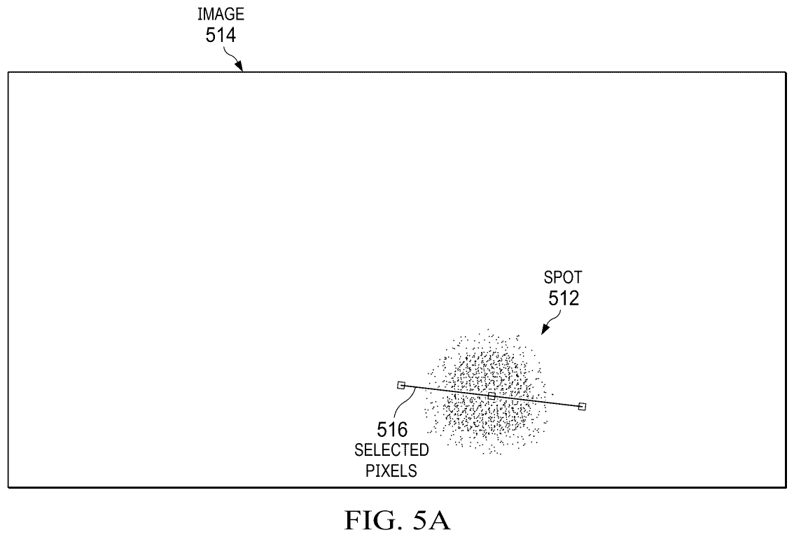

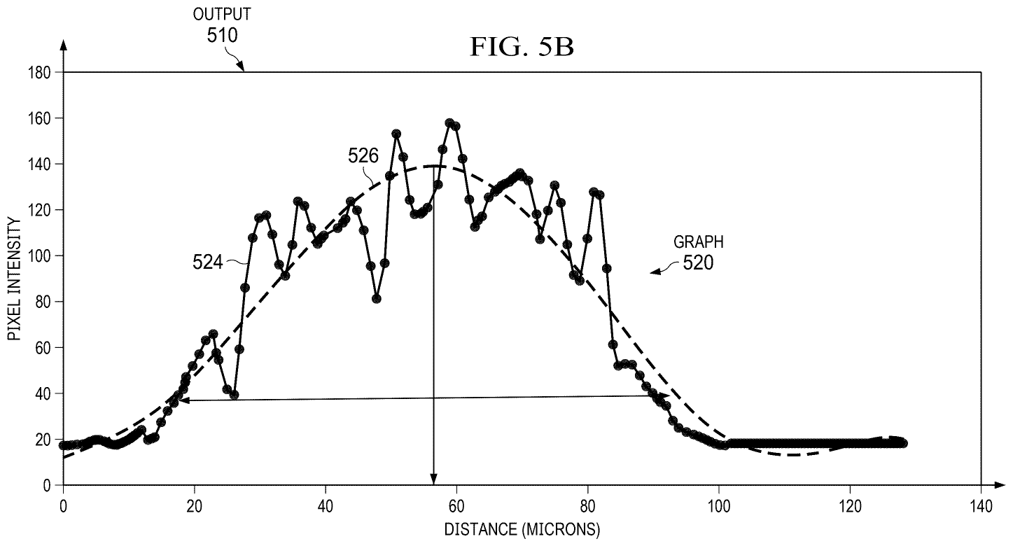

FIGS. 5A and 5B illustrate an example output 510 that describes an actual spot 512 in a digital image 514, according to at least one embodiment described in the present disclosure. FIG. 5A shows the digital image 514 with the spot 512 formed where light from a light system interacted with a test target. The digital image 514 shows selected pixels 516 that were selected to be analyzed. The selected pixels 516 may be automatically selected by a computer or may be selected by the user. For example, the computer may automatically select the pixels based on the expected size of the spot 512. As another example, the user may manipulate the graphical element representing the line elements of the selected pixels 516 in order to select the pixels.

FIG. 5B shows the output 510 of an analysis of the brightness of a light source of the light system. The output 510 includes a graph 520 with an x-axis that represents distance in microns and a y-axis that represents pixel intensity values (e.g., along a range from 0 to 255). The graph 520 shows the pixel intensity values 524 of the selected pixels and a best fit line 526 (e.g., a least squares line) of the pixel intensity values 524.

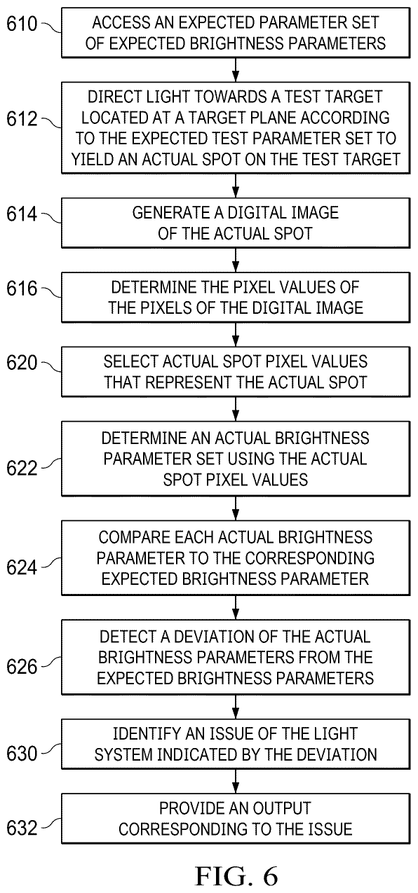

FIG. 6 illustrates an example of a method for detecting an issue with a light source, according to at least one embodiment described in the present disclosure. The method may be performed by any suitable system described herein.
At block 610, a computer accesses an expected parameter set of expected brightness parameters that describe the expected brightness of an actual spot.
At block 612, a light system directs light towards a test target located at a target plane according to the expected parameter set to yield the actual spot on the test target.
At block 614, an imaging system generates a digital image of the actual spot.
At block 616, the computer determines the pixel values of the pixels of the digital image.
At block 620, the computer selects actual spot pixel values that represent the actual spot from the pixel values.
At block 622, the computer determines an actual brightness parameter set of the actual spot using the actual spot pixel values. The actual brightness parameter set includes actual brightness parameters that describe the actual brightness of the actual spot. In certain embodiments, the computer may determine an actual brightness parameter according to pixel values that comprise grayscale values. For example, the computer may determine: an actual maximum brightness value according to the maximum grayscale value of the set; an actual average brightness value according to the average grayscale value of the set; an actual brightness statistical distribution value according to the grayscale statistical distribution value of the set; and/or an actual brightness distribution evenness value according to the grayscale distribution evenness value of the set. In certain embodiments, the computer may determine an actual brightness parameter according to pixel values that comprise color scale values. For example, the computer may determine an average color scale value of the set and/or a color scale distribution evenness value of the set.
At block 624, the computer compares each actual brightness parameter to the corresponding expected brightness parameter.
At block 626, the computer detects a deviation of the actual brightness parameters from the expected brightness parameters in response to the comparison.
At block 630, the computer identifies an issue of the light system indicated by the deviation(s).
At block 632, the computer provides an output corresponding to the issue. For example, the output may include the computer calculating a correction to compensate for the deviation and instructing the light system to implement the correction to compensate for the deviation. As another example of the output, the computer may provide a notification of a difference in brightness and/or color indicated by the issue. As another example of the output, the computer may provide a warning notification if an actual brightness value indicated by the one or more issues satisfies a warning range.

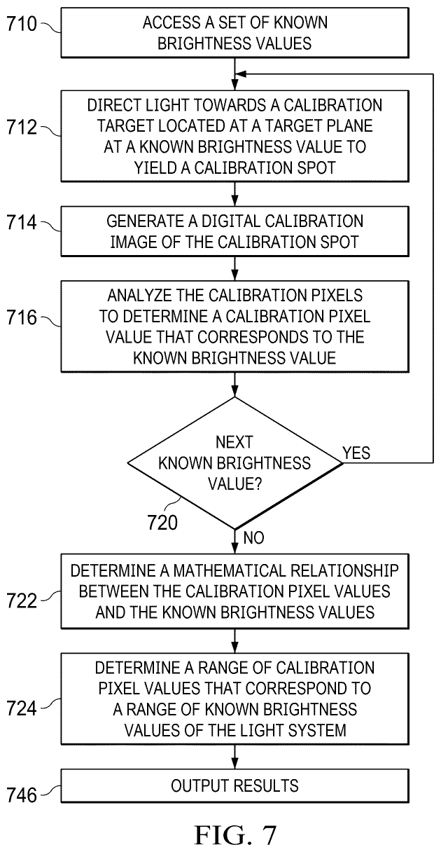

FIG. 7 illustrates an example of a calibration method, according to at least one embodiment described in the present disclosure. The method may be performed by any suitable system described herein.
At block 710, the computer accesses a set of known brightness values.
At block 712, the computer instructs the light system to direct light towards a calibration target located at the target plane at a known brightness value to yield a calibration spot on the calibration target.
At block 714, the computer instructs the imaging system to generate a digital calibration image of the calibration spot.
At block 716, the computer analyzes the calibration pixels of the digital calibration image to determine the calibration pixel value that corresponds to the known brightness value.
At block 720, there may be a next known brightness value of the set. If there is, the method returns to block 712 to process the next known brightness value of the set. If there is not, the method proceeds to block 722.
At block 722, the computer determines a mathematical relationship between the calibration pixel values and the known brightness values. For example, the computer may perform a least-squares fit of the values to yield a mathematical function f(P) =B that yields a brightness value B for a given pixel value P.
At block 724, the computer determines a range of calibration pixel values that correspond to a range of known brightness values. For example, the computer may determine a range of calibration pixel values that correspond to an expected range of brightness values. As another example, the computer may determine a range of calibration pixel values that correspond to a warning range of brightness values when the brightness values may initiate a warning by the system.
At block 726, the computer outputs the results, which may include, e.g., the mathematical relationship between the calibration pixel values and the known brightness values and/or the range of calibration pixel values that correspond to a range of known brightness values.

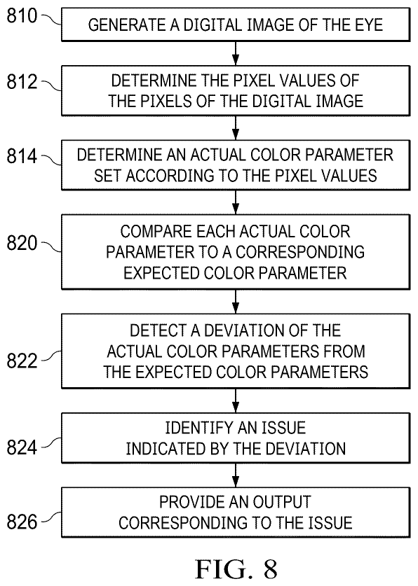

FIG. 8 illustrates an example of detecting an issue with an eye, according to at least one embodiment described in the present disclosure. The method may be performed by any suitable system described herein.
At block 810, an imaging system generates a digital image of the eye.
At block 812, the computer determines the pixel values of the pixels of the digital image.
At block 814, the computer determines an actual color parameter set according to the pixel values. The actual color parameter set includes actual color parameters describing the actual color of at least a portion of the actual eye, e.g., the iris and/or sclera.
At block 820, the computer compares each actual color parameter to a corresponding expected color parameter that describes an expected color of an expected eye.
At block 822, the computer detects a deviation of the actual color parameters from the expected color parameters.
At block 824, the computer identifies an issue indicated by the deviation. For example, the computer may detect a deviation that indicates that the actual eye is not the expected eye. For instance, a deviation in the coloration, the pattern of colors, or other suitable deviation of the actual eye may indicate that the actual eye is not the expected eye. As another example, the computer may detect a deviation that indicates that the actual eye has a medical condition. For instance, the coloration may indicate pigmentary glaucoma, Horner's syndrome, liver malfunctioning, pre-cancerous primary acquired melanosis (PAM), or any other disease detectable via coloration.
At block 826, the computer provides an output corresponding to the issue. For example, the computer provides a notification that the actual eye is not the expected eye or that the actual eye has a medical condition.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to”or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a”or “an”or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. § 112(f).

What is claimed:

1. An ophthalmic system, comprising:
a light system configured to:
direct light towards a test target located at a target plane according to an expected parameter set to yield at least one actual spot on the test target, the expected parameter set comprising one or more expected brightness parameters, each expected brightness parameter of the one or more expected brightness parameters describing an expected brightness of the at least one actual spot;
an imaging system comprising one or more digital cameras configured to generate a digital image of the least one actual spot; and
a computer configured to:
determine a plurality of pixel values of a plurality of pixels of the digital image;
determine an actual brightness parameter set according to the plurality of pixel values, the actual brightness parameter set comprising one or more actual brightness parameters, each actual brightness parameter of the one or more actual brightness parameters describing an actual brightness of the at least one actual spot, the each actual brightness parameter of the one or more actual brightness parameters corresponding to an expected brightness parameter of the one or more expected brightness parameters;
compare the each actual brightness parameter of the one or more actual brightness parameters to the corresponding expected brightness parameter of the one or more expected brightness parameters;
detect one or more deviations of the one or more actual brightness parameters from the one or more expected brightness parameters in response to the comparison;
identify one or more issues of the light system indicated by the one or more deviations; and
provide an output associated with the one or more issues of the light system.

2. The ophthalmic system of claim 1, the computer configured to determine the actual brightness parameter set according to the plurality of pixel values by:
selecting a plurality of actual spot pixel values from the plurality of pixel values, the plurality of actual spot pixel values representing the at least one actual spot; and
determining the actual brightness parameter set using the plurality of actual spot pixel values.

3. The ophthalmic system of claim 2, the computer configured to select the plurality of actual spot pixel values from the plurality of pixel values by:
identifying the plurality of actual spot pixel values according to an actual spot range of pixel values.

4. The ophthalmic system of claim 2, the computer configured to select the plurality of actual spot pixel values from the plurality of pixel values by:
identifying the plurality of actual spot pixel values according to a user selection.

5. The ophthalmic system of claim 1, the actual brightness parameter set comprising one or more elements selected from a group comprising: a maximum grayscale value, an actual maximum brightness value, an average grayscale value, an actual average brightness value, a grayscale statistical distribution value, an actual brightness statistical distribution value, a grayscale distribution evenness value, and an actual brightness distribution evenness value.

6. The ophthalmic system of claim 1, the plurality of pixel values comprising a plurality of grayscale values.

7. The ophthalmic system of claim 6, the computer configured to determine the actual brightness parameter set according to the plurality of pixel values by:
identifying a maximum grayscale value of the plurality of grayscale values; and
determining an actual maximum brightness value according to the maximum grayscale value of the plurality of grayscale values.

8. The ophthalmic system of claim 6, the computer configured to determine the actual brightness parameter set according to the plurality of pixel values by:
calculating an average grayscale value of the plurality of grayscale values; and
determining an actual average brightness value according to the average grayscale value of the plurality of grayscale values.

9. The ophthalmic system of claim 6, the computer configured to determine the actual brightness parameter set according to the plurality of pixel values by:
calculating a grayscale statistical distribution value of the plurality of grayscale values; and
determining an actual brightness statistical distribution value according to the grayscale statistical distribution value of the plurality of grayscale values.

10. The ophthalmic system of claim 6, the computer configured to determine the actual brightness parameter set according to the plurality of pixel values by:
calculating a grayscale distribution evenness value of the plurality of grayscale values; and
determining an actual brightness distribution evenness value according to the grayscale distribution evenness value of the plurality of grayscale values.

11. The ophthalmic system of claim 1:
the plurality of pixel values comprising a plurality of color scale values; and
the computer configured to determine an actual color value according to at least one color scale value of the plurality of color scale values.

12. The ophthalmic system of claim 11, the computer configured to determine the actual color value according to at least one color scale value of the plurality of color scale values by:
calculating an average color scale value of at least two color scale values of the plurality of color scale values.

13. The ophthalmic system of claim 11, the computer configured to determine the actual color value according to at least one color scale value of the plurality of color scale values by:
calculating a color scale distribution evenness value of at least two color scale values of the plurality of color scale values.

14. The ophthalmic system of claim 1, the computer configured to provide the output associated with the one or more issues of the light system by:
calculating a correction to compensate for a deviation of the one or more deviations; and
instructing the light system to implement the correction to compensate for the deviation of the one or more deviations.

15. The ophthalmic system of claim 1, the computer configured to provide the output associated with the one or more issues of the light system by:
providing a notification of a difference in brightness indicated by the one or more issues.

16. The ophthalmic system of claim 1, the computer configured to provide the output associated with the one or more issues of the light system by:
providing a notification of a difference in color indicated by the one or more issues.

17. The ophthalmic system of claim 1, the computer configured to provide the output associated with the one or more issues of the light system by:
providing a warning notification if an actual brightness parameter of the one or more actual brightness parameters satisfies a warning range.

18. The ophthalmic system of claim 1, the computer configured to provide the output associated with the one or more issues of the light system by:
providing a notification to a user via an interface.

19. The ophthalmic system of claim 1, the computer configured to provide the output associated with the one or more issues of the light system by:
sending a notification to a service technician.

20. The ophthalmic system of claim 1, the computer configured to:
record an actual brightness parameter of the one or more actual brightness parameters in a historical data set comprising a plurality of historical actual brightness parameters.

21. The ophthalmic system of claim 20, the computer configured to:
analyze the historical data set to determine a warning range indicating a range of actual brightness values that initiate a warning associated with the light system.

22. The ophthalmic system of claim 20, the computer configured to:
analyze the historical data set to determine an average lifespan of the light system; and
provide a reminder prior to an expiration of the average lifespan of the light system.

23. The ophthalmic system of claim 20, the computer configured to:
analyze the historical data set to detect a trend of an issue of the one or more issues of the light system.

24. The ophthalmic system of claim 1, the computer is configured to perform the following for each known brightness value of a plurality of known brightness values to yield a plurality of calibration pixel values, each calibration pixel value of the plurality of calibration pixel values corresponding to a known brightness value of the plurality of known brightness values:
instruct the light system to direct light towards a calibration target located at the target plane at a respective known brightness value of the plurality of known brightness values to yield a calibration spot on the calibration target;
instruct the imaging system to generate a digital calibration image of the calibration spot at the respective known brightness value, the digital calibration image comprising a plurality of calibration pixels; and
analyze the plurality of calibration pixels to determine a calibration pixel value of the plurality of calibration pixel values that corresponds to the respective known brightness value.

25. The ophthalmic system of claim 24, the computer configured to determine a mathematical relationship between the plurality of calibration pixel values and the plurality of known brightness values.

26. The ophthalmic system of claim 1, the light system comprising at least one system selected from a group comprising:
a laser system configured to direct a laser beam towards the target plane;
an illumination light system configured to direct an illumination beam towards the target plane to illuminate the target plane;
an aiming light system configured to direct an aiming beam towards the target plane to align the laser beam and the target plane; and
a fixation light system configured to direct a fixation beam towards the target plane to align an eye and the laser system.

27. An ophthalmic system, comprising:
an imaging system comprising one or more digital cameras configured to generate a digital image of an actual eye; and
a computer configured to:
determine a plurality of pixel values of a plurality of pixels of the digital image;
determine an actual color parameter set according to the plurality of pixel values, the actual color parameter set comprising one or more actual color parameters, each actual color parameter describing an actual color of the actual eye;
compare each actual color parameter of the one or more actual color parameters to a corresponding expected color parameter of one or more expected color parameters, each expected color parameter describing an expected color of an expected eye;
detect one or more deviations of the one or more actual color parameters from the one or more expected color parameters in response to the comparison;
identify one or more issues indicated by the one or more deviations; and
provide an output associated with the one or more issues.

28. The ophthalmic system of claim 27, the computer configured to identify the one or more issues indicated by the one or more deviations by:
determining that the actual eye is not the expected eye.

29. The ophthalmic system of claim 28, the computer configured to provide the output associated with the one or more issues by:
providing a notification that the actual eye is not the expected eye.

30. The ophthalmic system of claim 27, the computer configured to identify the one or more issues indicated by the one or more deviations by:
determining that the actual eye has a medical condition.

31. The ophthalmic system of claim 30, the computer configured to provide the output associated with the one or more issues by:
providing a notification that the actual eye has the medical condition.
